(19) United States
(12) Patent Application Publication
(10) Pub. No.: US 2012/0184537 A1
(43) Pub. Date: Jul. 19, 2012

(54) [1,4]-BENZODIAZEPINES AS VASOPRESSIN V2 RECEPTOR ANTAGONISTS

(75) Inventors: Ann Meulemans, Turnhout (BE); Karel Lavrijsen, Oud-Turnhout (BE); Marina Cools, Turnhout (BE); Erik Keller, Groningen (NL); Igor Alexnadrovich Mezine, Exton, PA (US); Leen Thielemans, Turnhout (BE); Maarten VAn Geffen, Turnhout (BE)

(73) Assignee: SHIRE-MOVETIS N.V.

(21) Appl. No.: 13/497,231

(22) PCT Filed: Sep. 23, 2010
(86) PCT No.: PCT/EP10/64044

§ 371 Date: Mar. 20, 2012

(30) Foreign Application Priority Data
Sep. 24, 2009 (GB) 916792.5
Aug. 16, 2010 (GB) 1013708.1

(51) Int. Cl. A61K 31/5513; C07D 243/14; A61P 9/12; A61P 9/04; A61P 7/10; A61P 9/10; A61P 1/16; A61P 13/12; A61P 25/00; A61P 7/02; C07D 498/04; A61P 9/00

(52) U.S. Cl. 514/220; 540/559; 540/573; 514/221

(57) ABSTRACT

The invention relates to a novel class of [1,4]-benzodiazepine derivatives, processes for their preparation, intermediates usable in these processes, and pharmaceutical compositions containing the compounds. Other aspects of the invention are directed to the use of said [1,4]-benzodiazepine derivatives in therapy based on the capability of said compounds to interfere with the binding of the peptide hormone, vasopressin, to its receptors. In particular as vasopressin V2 receptor antagonists and therefore useful for treating involving increased vascular resistance, cardiac insufficiency, and water retention.

### FIELD OF THE INVENTION

The invention relates to a novel class of [1,4]-benzodiazepine derivatives, processes for their preparation, intermediates usable in these processes, and pharmaceutical compositions containing the compounds. Other aspects of the invention are directed to the use of said [1,4]-benzodiazepine derivatives in therapy based on the capability of said compounds to interfere with the binding of the peptide hormone, vasopressin, to its receptors. In particular as vasopressin V2 receptor antagonists and therefore useful for treatments involving increased vascular resistance, cardiac insufficiency, and water retention.

### BACKGROUND TO THE INVENTION

The nonapeptide arginine vasopressin (AVP), which is principally secreted from the posterior pituitary gland, is responsible for numerous biological actions as both hormone and neurotransmitter. Three G-protein coupled receptors, denoted as V₁ₐ, V_{1b}, and V₂, are involved in AVP binding and cellular activation, resulting in important physiological responses such as reabsorption of water in the kidneys (V₂), contraction of the bladder, uterine, and vascular smooth muscle (V₁ₐ), breakdown of glycogen in the liver (V₁ₐ), aggregation of platelets (V₁ₐ), and release of corticotropin from the anterior pituitary gland (V_{1b}). Additionally, in the central nervous system, AVP modulates aggressive social, and sexual behaviour, stress response, and memory.

The V₂ receptors on renal epithelial cells mediate AVP-induced antidiuresis to preserve normal plasma osmolality. Thus, selective, nonapeptide vasopressin V₂ receptor antagonists have received attention for their potential use in treating diseases of excessive renal reabsorption of water. For example, the aquaretic effect of vasopressin V₂ receptor antagonists lead to a decreased peripheral resistance in conscious dogs with congestive heart failure (H. Ogawa, J. Med. Chem. 1996, 39, 3547). In certain pathological states, plasma vasopressin levels may be inappropriately elevated for a given osmolality, thereby resulting in renal water retention and hyponatremia. Hyponatremia, associated with edematous conditions (cirrhosis, congestive heart failure, renal failure), can be accompanied by the syndrome of inappropriate secretion of antidiuretic hormone (SIADH). Treatment of SIADH compromised rats with a vasopressin V-2 antagonist has corrected their existing hyponatremia (G. Fujisawa, Kidney Int. 1993, 44 (1), 19).

In view of the foregoing there is a continued need to find new therapeutic agents to treat vasopressin receptor mediated diseases, in particular V₂ receptor mediated diseases, and for treating conditions associated with such disorders.

### DETAILED DESCRIPTION OF THE INVENTION

It is accordingly an object of the present invention to provide a novel class of vasopressin receptor antagonists, characterized in having the formula (I)

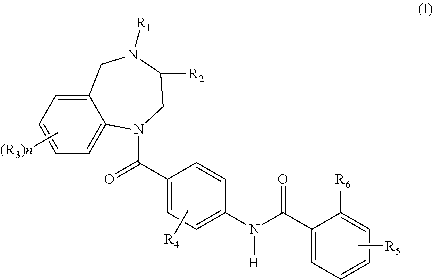

wherein;
n is 0, 1, 2 or 3;
R₁ is hydrogen; C₁₋₆alkenyl optionally substituted by one or more substituents selected from hydroxyl, halogen, nitro, amino, cyano, C₁₋₆alkoxy, hydroxycarbonyl, C₁₋₆alkoxycarbonyl or haloC₁₋₆alkoxy; or C₁₋₆alkyl substituted by one or more substituents selected from hydroxyl, halogen, nitro, amino, cyano, C₁₋₆alkoxy, hydroxycarbonyl, C₁₋₆alkoxycarbonyl or haloC₁₋₆alkoxy;
R₂ is C₁₋₆alkyl substituted by one or more substituents selected from hydroxyl, halogen, nitro, amino, cyano, C₁₋₆alkoxy, hydroxycarbonyl, C₁₋₆alkoxycarbonyl or haloC₁₋₆alkoxy; or
R₁ and R₂ taken together with the atom to which they are attached from a 6 membered heterocycle substituted with a substituent selected from oxo or hydroxyl;
R₃ is independently selected from hydrogen, hydroxyl, halogen, C₁₋₆alkoxy, C₁₋₆alkyl, or haloC₁₋₆alkoxy;
R₄ and R₅ are each independently selected from hydrogen, hydroxyl, halogen, C₁₋₆alkoxy, C₁₋₆alkyl, or haloC₁₋₆alkoxy;
R₆ is independently selected from phenyl or C₁₋₆alkyl;
and pharmaceutically acceptable enantiomers, racemates, diastereoisomers, solvates, hydrates, polymorphs and salts thereof.

The following are definitions of terms used in this specification and claims. The initial definition provided for a group or term herein applies to that group or term throughout the specification and claims, individually or as part of another group, unless otherwise indicated.
As used herein with respect to a substituting radical, and unless otherwise stated, the term “alkyl” relates to a fully saturated hydrocarbon; in this respect C₁₋₄alkyl defines straight and branched chain saturated hydrocarbon radicals having from 1 to 4 carbon atoms such as, for example, methyl, ethyl, propyl, butyl, 1-methylethyl, 2-methylpropyl, 2,2-dimethylethyl and the like; C₁₋₆alkyl is meant to include C₁₋₄alkyl and the higher homologues thereof having 5 or 6 carbon atoms such as, for example, pentyl, 2-methylbutyl, hexyl, 2-methylpentyl and the like.
As used herein with respect to a substituting radical, and unless otherwise stated, the term “alkenyl” means straight-chain, cyclic, or branched-chain hydrocarbon radicals containing at least one carbon-carbon double bond. Examples of alkenyl radicals include ethenyl, E- and Z-propenyl, isopropenyl, E- and Z-butenyl, E- and Z-isobutenyl, E- and Z-pentenyl, E- and Z-hexenyl, E,E-, E,Z-, Z,E-, Z,Z-hexadienyl, and the like. An optionally substituted alkenyl refers to an alkenyl having optionally one or more substituents (for example 1, 2, 3 or 4), selected from those defined above for the compounds of formula (I).
As used herein with respect to a substituting radical, and unless otherwise stated, the term “halo” or “halogen” refers to any atom selected from the group consisting of fluorine, chlorine, bromine and iodine.
As used herein with respect to a substituting radical, and unless otherwise stated, the term “heterocycle” is generic to substituted and unsubstituted non-aromatic 3 to 7 membered monocyclic groups, 7 to 11 membered bicyclic groups, and 10 to 15 membered tricyclic groups, in which at least one of the rings has at least one heteroatom (O, S or N). Each ring of the heterocyclo group containing a heteroatom can contain one or two oxygen or sulfur atoms and/or from one to four nitrogen atoms provided that the total number of heteroatoms in each ring is four or less, and further provided that the ring contains at least one carbon atom. The nitrogen and sulfur atoms may optionally be oxidized and the nitrogen atoms may optionally be quaternized. Exemplary heterocycles include pyrrolyl, pyrrolinyl, imidazolyl, imidazo linyl, pyrazolyl, pyrazolinyl, triazolyl, tetrazolyl, furanyl, tetrahydrofuranyl, thienyl, thiolanyl, dioxolanyl, oxazolyl, oxazolinyl, isoxazolyl, triazolyl, thiazolinyl, isothiazolyl, thiadiazolyl, oxadiazolyl, pyridinyl, pyrimidinyl, pyrazinyl, pyranyl, pyridazinyl, pyrrolidinyl, piperidinyl, piperazinyl, morpholinyl, thiomorpholinyl, dioxanyl, dithianyl, trithianyl, triazinyl, benzothienyl, isobenzothienyl, benzofuranyl, isobenzofuranyl, benzothiazolyl, benzoxazolyl, indolyl, isoindolyl, indolinyl, purinyl, 1H-pyrazolo[3,4-d]pyrimidinyl, benzimidazolyl, quinolyl, isoquinolyl, cinnolinyl, phtalazinyl, quinazolinyl, quinoxalinyl, thiazolopyridinyl, oxazolopyridinyl, and imidazo[2,1-b]thiazolyl. The heterocyclo group may be attached at any available nitrogen or carbon atom. Thus, for example, when the heterocycle is imidazolyl, it may be a 1-imidazolyl, 2-imidazolyl, 4-imidazolyl and 5-imidazolyl; when it is thiazolyl, it may be 2-thiazolyl, 4-thiazolyl and 5-thiazolyl; when it is triazolyl, it may be 1,2,4-triazolyl, 1,2,4-triazol-3-yl, 1,2,4-triazol-5-yl, 1,3,4-triazol-1-yl and 1,3,4-triazol-2-yl; when it is benzthiazolyl, it may be 2-benzthiazolyl, 4-benzthiazolyl, 5-benzthiazolyl, 6-benzthiazolyl and 7-benzthiazolyl. The heterocyclo groups are meant to include all the possible isomeric forms of the heterocycles mentioned in the definitions, for instance, pyrrolyl also includes 2H-pyrrolyl; triazolyl includes 1,2,4-triazolyl and 1,3,4-triazolyl; oxadiazolyl includes 1,2,3-oxadiazolyl, 1,2,4-oxadiazolyl, 1,2,5-oxadiazolyl and 1,3,4-oxadiazolyl; thiadiazolyl includes 1,2,3-thiadiazolyl, 1,2,4-thiadiazolyl, 1,2,5-thiadiazolyl and 1,3,4-thiadiazolyl; pyranyl includes 2H-pyranyl and 4H-pyranyl.
Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.
It will be appreciated that the structures depicted herein, and in particular the benzodiazepine moiety, includes partially unsaturated moieties, such as for example shown in example B.2.4. It is accordingly an object of the present invention to provide those compounds of formula (I) wherein the benzodiazepine is represented by the radical

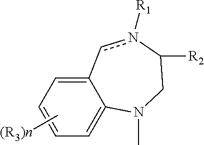

wherein n, R₁, R₂ and R₃ are as defined in the different embodiments of the present invention and

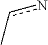

represent an optionally present double bound.
It will also be appreciated that the compounds of the present invention can exist in free form for treatment, or where appropriate, as a pharmaceutically acceptable salt, salts, or mixtures thereof.

As used herein, the term “pharmaceutically acceptable salt” refers to salts of a compound which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. These salts can be prepared in situ during the final isolation and purification of the compounds. Acid addition salts can be prepared by 1) reacting the purified compound in its free-based form with a suitable organic or inorganic acid and 2) isolating the salt thus formed.
Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersible products may be obtained by such quaternization.
Base addition salts can be prepared by 1) reacting the purified compound in its acid form with a suitable organic or inorganic base and 2) isolating the salt thus formed. Base addition salts include alkali or alkaline earth metal salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. Other acids and bases, while not in themselves pharmaceutically acceptable, may be employed in the preparation of salts useful as intermediates in obtaining the compounds of the invention and their pharmaceutically acceptable acid or base addition salts.

Examples of pharmaceutically acceptable salts also include internal salts such as N-oxides.

Where the compounds according to this invention have at least one stereogenic center, they may accordingly exist as enantiomers. As indicated in Formulae Ia and Ib, the compounds of interest to this invention have a specific absolute configuration at the stereocenter on the benzodiazepine ring, in the manner shown.

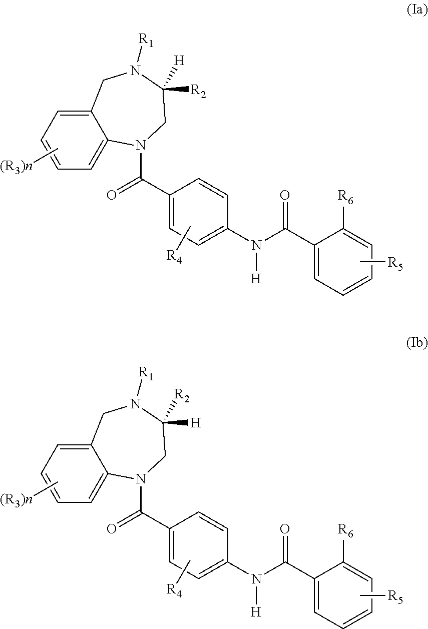

Where the compounds possess stereogenic centers in addition to this one, they may exist as diastereomers. It is to be understood that all such isomers and mixtures thereof are encompassed within the scope of the present invention. Furthermore, some of the crystalline forms for the compounds may exist as polymorphs and as such are intended to be included in the present invention.

In addition, some of the compounds of the present invention and their pharmaceutically acceptable salts (including quaternary derivatives and N-oxides) may form solvates with water (i.e., hydrates) or common organic solvents, and such solvates are also intended to be encompassed within the scope of this invention wherever a compound of the present invention or a salt thereof is herein referred to.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known in the art.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

In one embodiment, the present invention provides the compounds of formula (I), wherein R₁ is hydrogen or C₁₋₆alkyl substituted by one or more substituents selected from hydroxyl, halogen, nitro, amino, cyano, C₁₋₆alkoxy, hydroxycarbonyl, C₁₋₆alkoxycarbonyl or haloC₁₋₆alkoxy; in particular hydrogen or C₁₋₆alkyl substituted by hydroxyl, hydroxycarbonyl, or C₁₋₆alkoxycarbonyl; more in particular hydrogen or C₁₋₆alkyl substituted by hydroxycarbonyl, or C₁₋₆alkoxycarbonyl.

In one embodiment, the present invention provides the compounds of formula (I), wherein R₂ is hydrogen or C₁₋₆alkyl substituted by hydroxyl.

In another embodiment, the present invention provides the compounds of formula (I), wherein R₁ and R₂ taken together with the atom to which they are attached from a 6 membered heterocycle selected from piperidinyl, morpholinyl or thiomorpholinyl; in particular morpholinyl; substituted with oxo or hydroxyl. In a particular embodiment of the present invention said 6 membered heterocycle is substituted at position 6 of said morpholinyl or thiomorpholinyl.

In another embodiment, the present invention provides the compounds of formula (I), wherein n is 1 and R₃ is selected from hydrogen, halogen, or C₁₋₆alkyl; in particular n is 1 and R₃ is selected from hydrogen, halogen or methyl; more in particular n is 1 and R₃ is hydrogen; even more in particular n is 0.

In another embodiment, the present invention provides the compounds of formula (I), wherein R₄ is selected from hydrogen, hydroxyl, halogen, C₁₋₆alkoxy, or C₁₋₆alkyl; in particular R₄ is selected from hydroxyl, halogen, methyl or methoxy; more in particular R₄ is halogen; even more in particular R₄ is chloro. As is evident from the examples hereinafter, in a particular embodiment of the present invention said R₄ substituent is at the ortho position vis-à-vis the benzodiazepine part of the molecule.

In another embodiment, the present invention provides the compounds of formula (I), wherein R₅ is selected from hydrogen, hydroxyl, halogen, C₁₋₆alkoxy, or C₁₋₆alkyl; in particular R₅ is selected from hydrogen, hydroxyl, halogen, or methoxy; more in particular R₅ is hydrogen. In a particular embodiment said R₅ substituent is at position 4 or 5 of said phenylamide.

In another embodiment, the present invention provides the compounds of formula (I), wherein R₆ is phenyl.

As will be apparent to the skilled artisan, and evident from the exemplified compounds hereinafter, none of the aforementioned embodiments of the compounds of formula (I) should be considered in isolation. Further embodiments of the present invention include voluntary combinations of any one of the aforementioned embodiments.

Hence in a further embodiment the present invention provides those compounds of formula (I) wherein one or more of the following restrictions apply:
R₁ is hydrogen or C₁₋₆alkyl substituted by one or more substituents selected from hydroxyl, halogen, nitro, amino, cyano, C₁₋₆alkoxy, hydroxycarbonyl, C₁₋₆alkoxycarbonyl or haloC₁₋₆alkoxy; in particular hydrogen or C₁₋₆alkyl substituted by hydroxyl, hydroxycarbonyl, or C₁₋₆alkoxycarbonyl; more in particular hydrogen or C₁₋₆alkyl substituted by hydroxycarbonyl, or C₁₋₆alkoxycarbonyl.
R₂ is hydrogen or C₁₋₆alkyl substituted by hydroxyl.
R₁ and R₂ taken together with the atom to which they are attached from a 6 membered heterocycle selected from piperidinyl, morpholinyl or thiomorpholinyl; in particular morpholinyl; substituted with oxo or hydroxyl. In a particular embodiment of the present invention said 6 membered heterocycle is substituted at position 6 of said morpholinyl or thiomorpholinyl.
n is 1 and R₃ is selected from hydrogen, halogen, or C₁₋₆alkyl; in particular n is 1 and R₃ is selected from hydrogen, halogen or methyl; more in particular n is 1 and R₃ is hydrogen; even more in particular n is 0.
R₄ is selected from hydrogen, hydroxyl, halogen, C₁₋₆alkoxy, or C₁₋₆alkyl; in particular R₄ is selected from hydroxyl, halogen, methyl or methoxy; more in particular R₄ is halogen; even more in particular R₄ is chloro.
R₅ is selected from hydrogen, hydroxyl, halogen, C₁₋₆alkoxy, or C₁₋₆alkyl; in particular R₅ is selected from hydrogen, hydroxyl, halogen, or methoxy; more in particular R₅ is hydrogen. In a particular embodiment said R₅ substituent is at position 4 or 5 of said phenylamide.
R₆ is phenyl

The present invention further provides the compounds of formula (Ic)

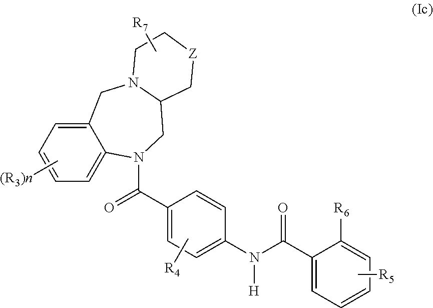

wherein;
n is 1, 2 or 3;
Z is O or S; in particular Z is 0;
R₃ is independently selected from hydrogen, hydroxyl, halogen, C₁₋₆alkoxy, C₁₋₆alkyl, or haloC₁₋₆alkoxy;
R₄ and R₅ are each independently selected from hydrogen, hydroxyl, halogen, C₁₋₆alkoxy, C₁₋₆alkyl, or haloC₁₋₆alkoxy;
R₆ is independently selected from phenyl or C₁₋₆alkyl;
R₇ is oxo or hydroxyl;
and pharmaceutically acceptable enantiomers, racemates, diastereoisomers, solvates, hydrates, polymorphs and salts thereof.

In another embodiment, the present invention provides the compounds of formula (Ic), wherein n is 1 and R₃ is selected from hydrogen, halogen, or C₁₋₆alkyl; in particular n is 1 and R₃ is selected from hydrogen, halogen or methyl; more in particular n is 1 and R₃ is hydrogen; even more in particular n is 0.

In another embodiment, the present invention provides the compounds of formula (Ic), wherein R₄ is selected from hydrogen, hydroxyl, halogen, C₁₋₆alkoxy, or C₁₋₆alkyl; in particular R₄ is selected from hydroxyl, halogen, methyl or methoxy; more in particular R₄ is halogen; even more in particular R₄ is chloro. As is evident from the examples hereinafter, in a particular embodiment of the present invention said R₄ substituent is at the ortho position vis-à-vis the benzodiazepine part of the molecule.

In another embodiment, the present invention provides the compounds of formula (Ic), wherein R₅ is selected from hydrogen, hydroxyl, halogen, C₁₋₆alkoxy, or C₁₋₆alkyl; in particular R₅ is selected from hydrogen, hydroxyl, halogen, or methoxy; more in particular R₅ is hydrogen. In a particular embodiment said R₅ substituent is at position 4 or 5 of said phenylamide.

In another embodiment, the present invention provides the compounds of formula (Ic), wherein R₆ is phenyl.

As will be apparent to the skilled artisan, and evident from the exemplified compounds hereinafter, none of the aforementioned embodiments of the compounds of formula (Ic) should be considered in isolation. Further embodiments of the present invention include voluntary combinations of any one of the aforementioned embodiments.

Hence in a further embodiment the present invention provides those compounds of formula (Ic) wherein one or more of the following restrictions apply:
n is 1 and R₃ is selected from hydrogen, halogen, or C₁₋₆alkyl; in particular n is 1 and R₃ is selected from hydrogen, halogen or methyl; more in particular n is 1 and R₃ is hydrogen; even more in particular n is 0.
R₄ is selected from hydrogen, hydroxyl, halogen, C₁₋₆alkoxy, or C₁₋₆alkyl; in particular R₄ is selected from hydroxyl, halogen, methyl or methoxy; more in particular R₄ is halogen; even more in particular R₄ is chloro. As is evident from the examples hereinafter, in a particular embodiment of the present invention said R₄ substituent is at the ortho position vis-à-vis the benzodiazepine part of the molecule.
R₅ is selected from hydrogen, hydroxyl, halogen, C₁₋₆alkoxy, or C₁₋₆alkyl; in particular R₅ is selected from hydrogen, hydroxyl, halogen, or methoxy; more in particular R₅ is hydrogen. In a particular embodiment said R₅ substituent is at position 4 or 5 of said phenylamide.
R₆ is phenyl.

The present invention further provides a composition comprising a compound of formula (I) or (Ic) suitable for use as a vasopressin receptor antagonist.

The present invention further provides a compound of formula (I) or (Ic); or a composition comprising a compound of formula (I) or (Ic); for use as a medicine.

The present invention further provides a compound of formula (I) or (Ic); or a composition comprising a compound of formula (I) or (Ic); for use in the treatment of a vasopressin V2 receptor mediated disorder; in particular said vasopressin V2 receptor mediated disorder is selected from the group consisting of hypertension, hyponatremia, congestive heart failure, cardiac insufficiency, coronary vasospasm, cardiac ischemia, liver cirrhosis, renal vasospasm, renal failure, diabetic nephropathy, polycystic kidney disease, cerebral edema and ischemia, stroke, thrombosis, and water retention.

Preferably, the V2 receptor mediated disorder is selected from hypertension, congestive heart failure, cardiac insufficiency, and hyponatremia.

The present invention further provides the use of a compound of formula (I) or (Ic); or a composition comprising a compound of formula (I) or (Ic); in the manufacture of a medicament for the treatment of a vasopressin V2 receptor mediated disorder.

The present invention further provides a method of treatment of a vasopressin V2 receptor mediated disorder, said method comprising administering to a subject in need thereof, a therapeutically effective amount of a compound of formula (I) or (Ic); or a composition comprising a compound of formula (I) or (Ic).

### Methods of Preparation

The compounds of this invention may be prepared in general by methods such as those depicted in the general schemes below, and the preparative examples that follow.

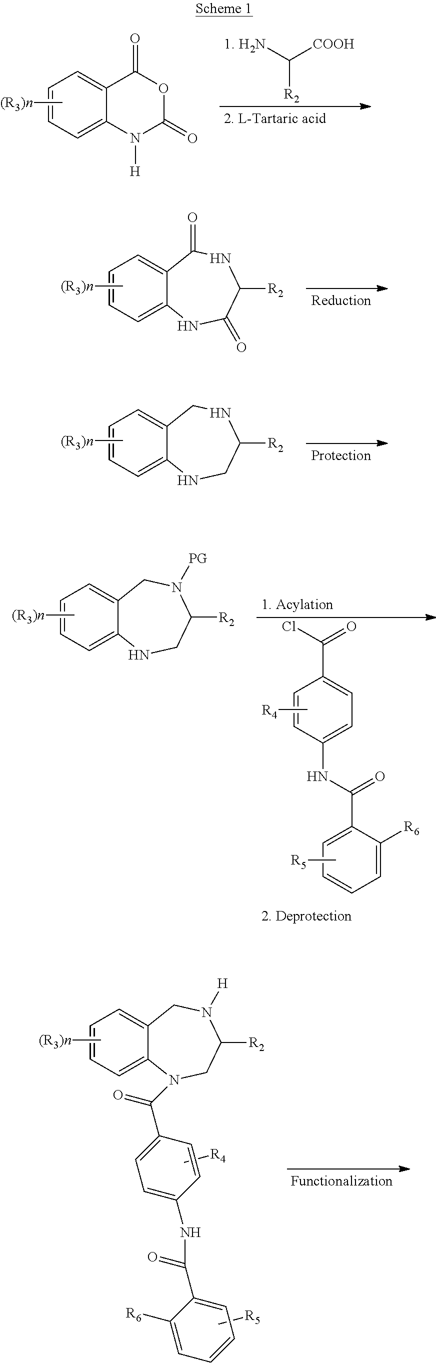

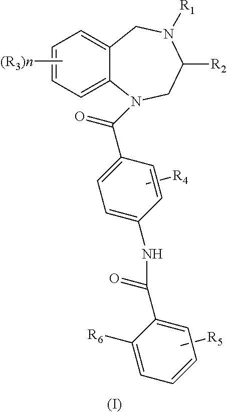

Scheme 1 provides a general synthesis scheme for the compounds of the present invention. Since this scheme is an illustration whereby intermediate and target compounds of the present may be prepared, the invention should not be construed as being limited by the chemical reactions and conditions expressed. The preparation of the various starting materials used in the scheme is well within the skill of persons versed in the art.

In Scheme 1, an R₃-substituted isatoic anhydride of formula (available either commercially or prepared by protocols reported in the scientific literature) may be condensed with an appropriate amino acid (1.) comprising the R₂ substituent of the compounds of the present invention, under basic conditions with heat. Subsequent addition of L-tartaric acid with continued heating provides R₂ substituted benzodiazepine-diones. One versed in the art will recognize that depending on the stereochemistry of the amino acid (1.) compounds of Scheme 1 and of the present invention can be synthesized with the opposite stereochemistry.

The thus obtained R₂ substituted benzodiazepine-diones may be reduced in the presence of a hydride source such as lithium aluminum hydride or the like in anhydrous ether solvent and subsequently protected with an appropriate amino protecting group (PG). At this stage, the available amino group may be acylated with a compound as presented. Subsequent removal of the amino protecting group (PG) by conventional methods gives compounds that still needs to be functionalized with the R₁ substituent as defined herein. For example, R₁ may be installed via sulfonylation, acylation, or reductive amination.

### Therapeutic Application

In a further embodiment the present invention provides the compounds of formula (I), (Ia), (Ib), or (Ic) for use as a medicine; in particular for use in the treatment of vasopressin receptor mediated disorders, such as for example hypertension, hyponatremia, congestive heart failure, cardiac insufficiency, coronary vasospasm, cardiac ischemia, liver cirrhosis, renal vasospasm, renal failure, diabetic nephropathy, polycystic kidney disease, cerebral edema and ischemia, stroke, thrombosis, and water retention.

As shown in the examples hereinafter, the compounds of the present invention are particularly useful as vasopressin V2 receptor antagonists and accordingly useful in the treatment of vasopressin V2 receptor mediated disorders. It is thus an object of the present invention to provide the use of the compounds as defined herein in the manufacture of a medicament for the treatment, amelioration or prevention of vasopressin V2 receptor mediated disorder, including but not limited to hypertension, hyponatremia, congestive heart failure, cardiac insufficiency, coronary vasospasm, cardiac ischemia, liver cirrhosis, renal vasospasm, renal failure, diabetic nephropathy, polycystic kidney disease, cerebral edema and ischemia, stroke, thrombosis, and water retention.

In view of the utility of the compounds according to the invention, there is provided a method for the treatment of an animal, for example, a mammal including humans, suffering from vasopressin V2 receptor mediated disorders, which comprises administering an effective amount of a compound according to the present invention.

Said method comprising the systemic or topical administration of an effective amount of a compound according to the invention, to animals, including humans.

The effective amount of a compound of formula (I), (Ia), (Ib), or (Ic) according to the present invention, also referred to here as the active ingredient, which is required to achieve a therapeutical effect will, of course, vary with the particular compound, the route of administration, the age and condition of the recipient, and the particular disorder or disease being treated. A person of ordinary skill in the art can easily determine an appropriate dose of compounds of the invention to administer to a subject without undue experimentation. Typically, a physician will determine the actual dosage that will be most suitable for an individual subject based upon a variety of factors including the activity of the specific compound employed, the metabolic stability and length of action of the compound, the age, body weight, general health, diet, mode and time of administration, rate of excretion, drug combination, the severity of the particular condition, and the individual undergoing therapy. To determine a suitable dose, the physician or veterinarian could start doses of a vasopressin receptor antagonist of the present invention at levels lower than that required in order to achieve the desired therapeutic effect and gradually increase the dosage until the desired effect is achieved. This is considered to be within the skill of the artisan and one can review the existing literature on a specific agent to determine optimal dosing. A suitable daily dose would range from 0.01 mg/kg to 300 mg/kg body weight, in particular from 0.5 mg/kg to 5.0 mg/kg body weight, more in particular from 1.0 mg/kg to 3.0 mg/kg body weight. A method of treatment may also include administering the active ingredient on a regimen of between one and four intakes per day.

The daily dose of a pharmaceutical composition of the present invention may be varied over a wide range from about 0.1 to 20,000 mg per adult human per day, however the dose will preferably be in the range of from about 1 to about 1,000 mg per adult human per day. For oral administration, the compositions are preferably provided in the form of tablets containing 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the subject to be treated. An effective amount of the drug is ordinarily supplied at a dosage level of from about 0.01 mg/kg/day to about 300 mg/kg/day. Particularly, the range is from about 0.5 to about 5.0 mg/kg of body weight per day; and more particularly, from about 1.0 to about 3.0 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level.

As another aspect of the present invention a combination of a vasopressin receptor antagonist with another agent used in the treatment of vasopressin receptor mediated disorders is envisaged, in particular the combination of vasopressin receptor antagonists with diuretics is envisaged.

Diuretics are commonly used in the treatment of hypertension and management of edema, such as with congestive heart failure. Of significant concern in such treatments is the loss of ions or electrolytes, particularly including sodium and potassium, with the increased volume of urine. The combination of a vasopressin receptor antagonist of the present invention with a diuretic not only increases urine flow, but also provides an improved method of retaining electrolytes or ions in the blood during diuretic administration.

Among the diuretic agents useful for the combination regimens of this invention are thiazide and related sulfonamide diuretics bendroflumethiazide, benzthiazide, chlorothiazide, chlorthalidone, cyclothiazide, hydrochlorothiazide, hydroflumethiazide, indapamide, methylclothiazide, metolazone, polythiazide, quinethazone and thrichlormethiazide. Also useful are potassium-sparing diuretics, such as amiloride, spironolactone and triamterene. Among the more preferred diuretics for use with this invention are the Loop diuretics, such as bumetanide, ethacrynic acid, ethacrynate sodium, and furosemide (sold under the Lasix® tradename, Hoechst Marion Roussel). The diuretics herein are known in the art and can be administered in the fashion and at the concentrations known in the art.

While it is possible for the active ingredient to be administered alone, it is preferable to present it as a composition.

### Pharmaceutical Compositions

It is also an object of the present invention to provide a composition comprising a vasopressin receptor antagonist as defined hereinbefore, suitable for use in treating and/or preventing vasopressin receptor mediated diseases in a subject in need thereof.

The pharmaceutical compositions of the present invention can be prepared by any known or otherwise effective method for formulating or manufacturing the selected product form. Methods of formulating pharmaceutical compositions have been described in numerous publications such as Pharmaceutical Dosage Forms Tablets, Second Edition, Revised and Expanded. Volumes 1-3, edited by Lieberman et al; Pharmaceutical Dosage Forms: Parenteral Medications, Volumes 1-2, edited by Avis et al; and Pharmaceutical Dosage Forms: Disperse Systems. Volumes 1-2, edited by Lieberman et al; published by Marcel Dekker, Inc. In preparing a pharmaceutical composition of the present invention in liquid dosage form for oral, topical and parenteral administration, any of the usual pharmaceutical media or excipients may be employed. For example, vasopressin receptor modulators can be formulated along with common excipients, diluents, or carriers, and formed into oral tablets, capsules, sprays, mouth washes, lozenges, treated substrates (e.g., oral or topical swabs, pads, or disposable, non-digestible substrate treated with the compositions of the present invention); oral liquids (e.g., suspensions, solutions, emulsions), powders, or any other suitable dosage form.

Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain. Non-limiting examples of suitable excipients, diluents, and carriers include: fillers and extenders such as starch, sugars, mannitol, and silicic derivatives; binding agents such as carboxymethyl cellulose and other cellulose derivatives, alginates, gelatin, and polyvinyl pyrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as acetyl alcohol, glycerol monostearate; adsorptive carriers such as kaolin and bentonite; carriers such as propylene glycol and ethyl alcohol, and lubricants such as talc, calcium and magnesium stearate, and solid polyethyl glycols.

The compounds according to the invention can also be formulated as elixirs or solutions for convenient oral administration or as solutions appropriate for parenteral administration, for instance by intramuscular, subcutaneous or intravenous routes. Additionally, the vasopressin receptor inhibitors are also well suited for formulation as a sustained or prolonged release dosage forms, including dosage forms that release active ingredient only or preferably in a particular part of the intestinal tract, preferably over an extended or prolonged period of time to further enhance effectiveness. The coatings, envelopes, and protective matrices in such dosage forms may be made, for example, from polymeric substances or waxes well known in the pharmaceutical arts.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

The pharmaceutical compositions herein will contain, per dosage unit, e.g., tablet, capsule, powder, injection, teaspoonful and the like, an amount of the active ingredient necessary to deliver an effective dose as described above. The therapeutically effective amount of the compounds of Formula (I) exemplified in such a method is from about 0.01 mg/kg/day to about 300 mg/kg/day. Particularly, the range is from about 0.5 to about 5.0 mg/kg of body weight per day; and more particularly, from about 1.0 to about 3.0 mg/kg of body weight per day. The compounds may be administered on a regimen of 1 to 4 times per day. Preferably, for the method of treating vascular resistance disorders described in the present invention using any of the compounds as defined herein, the dosage form will contain a pharmaceutically acceptable carrier containing between about 0.01 mg and 100 mg, more preferably about 5 to 50 mg, of the compound, and may be constituted into any form suitable for the mode of administration selected. The dosages, however, may be varied depending upon the requirement of the patients, the severity of the condition being treated and the compound being employed. The use of either daily administration or post-periodic dosing may be employed.

Formulations suitable for oral administration may be in the form of capsules, cachets, pills, tablets, lozenges (using a flavored basis, usually sucrose and acacia or tragacanth), powders, granules, or as a solution or a suspension in an aqueous or nonaqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents, in the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsiflers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

For nasal administration, the compounds according to the invention may be dissolved in a physiologically acceptable pharmaceutical carrier and administered as a solution or spray. Illustrative of suitable pharmaceutical carriers are water, saline, and aqueous alcoholic solutions. The pharmaceutical carrier may also contain preservatives, buffers, or other material suitable for such a dosage form.

For inhalation therapy, the compounds according to the invention can be incorporated into an aqueous alcoholic solution containing a fluorinated hydrocarbon propellant and packaged into a suitable administration device as known in the art.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

### EXAMPLES

### A. In Vitro Assay

### A.1. In Vitro Recombinant Vasopressin Receptor Binding Assay

The in vitro inhibition of AVP receptor binding was assessed by using recombinant human V₁ₐ or V₂ receptor preparations derived from the membranes of transfected CHO cells. Compounds were assessed for their ability to displace [³H]-arginine vasopressin from plasma membrane preparations of said CHO transfected cells in analogy with the Binding Assay procedures described in Cotte N., et al., (1998) J. Biol. Chem., 273; 29462-29468 and Tahara A., et al., (1998) Brit. J. Pharmacol., 125; 1463-1470; which are incorporated herein by reference.

### A.1.1. Human Vasopressin V₂ Receptor Binding Assay

Cell membrane homogenates (16 μg protein) are incubated for 120 min at 22° C. with 0.3 nM [³H]AVP in the absence or presence of the test compound in a buffer containing 50 mM Tris-HCl (pH 7.4), 5 mM MgCl₂ and 0.1% BSA. Nonspecific binding is determined in the presence of 1 μM AVP. Following incubation, the samples are filtered rapidly under vacuum through glass fiber filters (GF/B, Packard) presoaked with 0.3% PEI and rinsed three times with ice-cold 50 mM Tris-HCl using a 96-sample cell harvester (Unifilter, Packard). The filters are dried then counted for radioactivity in a scintillation counter (Topcount, Packard) using a scintillation cocktail (Microscint 0, Packard).

### A.1.2. Human Vasopressin V₁ₐ Receptor Binding Assay

Cell membrane homogenates (40 μg protein) are incubated for min at 22° C. with 0.3 nM [³H]AVP in the absence or presence of the test compound in a buffer containing 50 mM Tris-HCl (pH 7.4), 5 mM MgCl₂ and 0.1% BSA. Nonspecific binding is determined in the presence of 1 μM AVP. Following incubation, the samples are filtered rapidly under vacuum through glass fiber filters (GF/B, Packard) presoaked with 0.3% PEI and rinsed three times with ice-cold 50 mM Tris-HCl using a 96-sample cell harvester (Unifilter, Packard). The filters are dried then counted for radioactivity in a scintillation counter (Topcount, Packard) using a scintillation cocktail (Microscint 0, Packard).

| | V1a | V2 |
|---|---|---|
| | IC₅₀ | IC₅₀ |
| Compound | (nM) | (nM) |
| 14 | 3800 | 5.8 |
| 16 | 240 | 1.9 |
| 17 | — | 120 |
| 19 | 210 | 2.5 |
| 21 | 780 | 19 |

The standard reference compound is AVP (V₂ receptor binding assay) or [d(CH2)51,Tyr(Me)2]-AVP (V₁ₐ receptor binding assay), which is tested in each experiment at several concentrations to obtain a competition curve from which its IC₅₀ is calculated.

### A.2. In Vitro Inhibition of AVP-Induced Effects in Human Vasopressin Receptors

### A.2.1. Human V₂ Receptor

The accumulation of cAMP was measured in transfected CHO cells (supra) expressing the human V2 receptor in analogy with the cAMP Assay procedures described in Cotte N., et al., (1998) J. Biol. Chem., 273; 29462-29468; which is incorporated herein by reference.

### A.2.1.1. Human V₂ Agonist Effect

The cells are suspended in HBSS buffer (Invitrogen) complemented with 20 mM HEPES 20 (pH 7.4), 0.01% BSA and 500 μM IBMX, then distributed in microplates at a density of 3×103 cells/well and incubated for 30 min at 22° C. in the absence (control) or presence of the test compound or the reference agonist. For stimulated control measurement, separate assay wells contain AVP at a final concentration of 1 nM. Following incubation, the cells are lysed and the fluorescence acceptor (D2-labeled cAMP) and fluorescence donor (anti-cAMP antibody labeled with europium cryptate) are added. After 60 min at 22° C., the fluorescence transfer is measured at λex=337 nm and λem=620 and 665 nm using a microplate reader (Rubystar, BMG). The cAMP concentration is determined by dividing the signal measured at 665 nm by that measured at 620 nm (ratio).

The results are expressed as a percent of the control response to 1 nM AVP.

### A.2.1.2. Human V₂ Antagonist Effect

The cells are suspended in HBSS buffer (Invitrogen) complemented with 20 mM HEPES (pH 7.4), 0.01% and 500 μM IBMX, then distributed in microplates at a density of 3×103 cells/well and preincubated for 5 min at 22° C. in the absence (control) or presence of the test compound or the reference antagonist. Thereafter, the reference agonist AVP is added at a final concentration of 0.03 nM. For basal control measurements, separate assay wells do not contain AVP.

Following 30 min incubation at 22° C., the cells are lysed and the fluorescence acceptor (D2-labeled cAMP) and fluorescence donor (anti-cAMP antibody labeled with europium cryptate) are added. After 60 min at 22° C., the fluorescence transfer is measured at λex=337 nm and λem=620 and 665 nm using a microplate reader (Rubystar, BMG). The cAMP concentration is determined by dividing the signal measured at 665 nm by that measured at 620 nm (ratio). The results are expressed as a percent inhibition of the control response to 0.03 nM AVP.

The standard reference antagonist is [adamantaneacetyl¹,O-Et-D-Tyr²,Val⁴,aminobutyryl⁶]-AVP, which is tested in each experiment at several concentrations to generate a concentration-response curve from which its IC₅₀ value is calculated.

### A.2.2. Human V₁ₐ Receptor

Intracellular calcium mobilization was measured in CHO cells transfected to express either human V1a receptors (supra), in analogy with the Measurement of intracellular Ca²⁺ concentration as described in Tahara A., et al., (1998) Brit. J. Pharmacol., 125; 1463-1470; which is incorporated herein by reference.

### A.2.2.1. Human V₁ₐ Agonist Effect

The cells are suspended in DMEM buffer (Invitrogen) complemented with 0.1% fetal calf serum, then distributed in microplates at a density of 4.5×10⁴ cells/well.

The fluorescent probe (Calcium4, Molecular Device) mixed with probenicid in HBSS buffer (Invitrogen) complemented with 20 mM Hepes (Invitrogen) (pH 7.4) is then added into each well and equilibrated with the cells for 60 min at 37° C. then 15 min at 22° C. Thereafter, the assay plates are positioned in a microplate reader (CellLux, PerkinElmer) which is used for the addition of the test compound, reference agonist or HBSS buffer (basal control), and for the measurements of changes in fluorescence intensity which varies proportionally to the free cytosolic Ca²⁺ ion concentration. For stimulated control measurements, AVP at 1 μM is added in separate assay wells.

The results are expressed as a percent of the control response to 1 μM AVP.

### A.2.2.2. Human V₁ₐ Antagonist Effect

The cells are suspended in DMEM buffer (Invitrogen) complemented with 0.1% fetal calf serum, then distributed in microplates at a density of 4.5×104 cells/well.

The fluorescent probe (Calcium4, Molecular Device) mixed with probenicid in HBSS buffer (Invitrogen) complemented with 20 mM Hepes (Invitrogen) (pH 7.4) is then added into each well and equilibrated with the cells for 60 min at 37° C. then 15 min at 22° C. Thereafter, the assay plates are positioned in a microplate reader (CellLux, PerkinElmer) which is used for the addition of the test compound or HBSS buffer then 5 min later 10 nM AVP or HBSS buffer (control), and for the measurements of changes in fluorescence intensity which varies proportionally to the free cytosolic Ca2+ ion concentration.

The standard reference antagonist is [d(CH₂)⁵¹,Tyr(Me)²,Arg⁸]-AVP, which is tested in each experiment at several concentrations to generate a concentration-response curve from which its IC₅₀ value is calculated.

### A.2.3. Results Agonist Effect

| Compound | | |
|---|---|---|
| at 1 μM | V1a | V2 |
| 14 | −1 | −1 |
| 16 | −1 | 0 |
| 17 | −1 | −1 |
| 19 | −1 | 0 |
| 21 | — | −1.5 |

Results are expressed as % of control agonist response (AVP) at a test concentration of 1 μM and 1 nM respectively.

As is evident from these results, the compounds of the present invention showed no agonstic response in the aforementioned assays.

### A.2.4. Results Antagonist Effect

| | V1a | V2 |
|---|---|---|
| | IC₅₀ | IC₅₀ |
| Compound | (nM) | (nM) |
| 14 | >100000 | 57 |
| 16 | >100000 | 13 |
| 17 | — | 2800 |
| 19 | 20000 | 11 |
| 21 | >100000 | 410 |

Results of antagonism are expressed as IC₅₀ values in nM.

### B. Compound Synthesis

The compounds of the invention may be prepared by methods well known to those skilled in the art, and as described in the synthetic and experimental procedures shown below.

### B.1. Synthesis of Intermediates

### B.1.1. Preparation of (S)-tert-butyl 3-(hydroxymethyl)-2,3-dihydro-1H-benzo[e][1,4]diazepine-4(5H)-carboxylate (2)

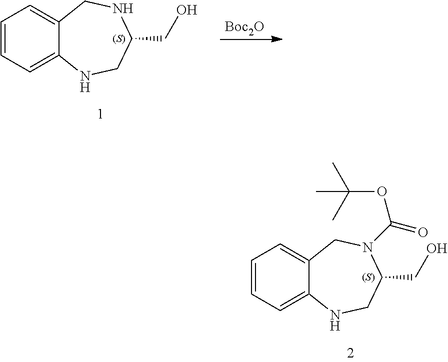

3-(S)-hydroxymethyl-2,3,4,5-tetrahydro-1,4-benzodiazepine (1) (CAS N^{o} 886225-46-3) (95 g, 0.378 mol, 1.0 equiv), Boc₂O (248 g, 0.946 mol, 2.5 equiv) and Et₃N (131 ml, 1.135 mol, 3.0 equiv) were added to a 1:1 mixture of water (500 ml) and EtOAc (500 ml). The mixture was heated to reflux (overnight). The mixture was allowed to cool to RT before separating the layers. The organic layer was washed with 25% NH₃ (aq) (2×500 ml). The organic layer was dried (Na₂SO₄) and concentrated, yielding (S)-tert-butyl 3-(hydroxymethyl)-2,3-dihydro-1H-benzo[e][1,4]diazepine-4(5H)-carboxylate (2) (Yield: 98.7 g (beige solid), 94%).

### Analytical Data

¹H-NMR (300 MHz, CDCl₃): 1.35 (s, 9H), 3.22 (dd, 1H), 3.50 (q, 1H), 3.76 (d, 2H), 4.43 (q, 3H), 6.59 (d, 1H), 6.72 (t, 1H), 6.98 (t, 2H)

### B.1.2. Preparation of 4-biphenyl-2-ylcarboxamido-2-chlorobenzoic acid (7)

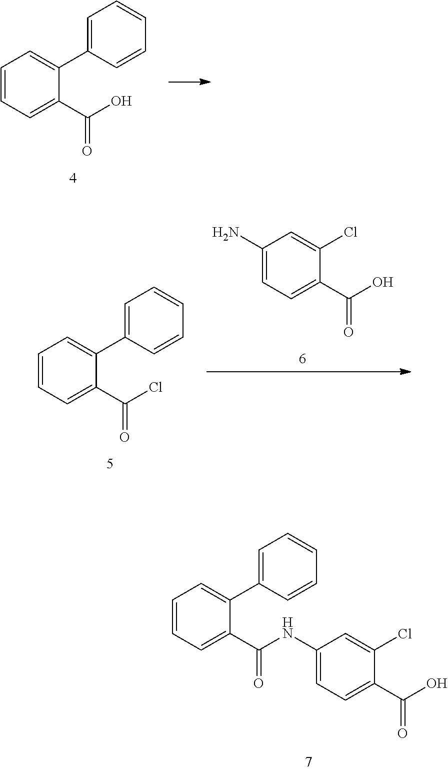

2-phenylbenzoic acid (4) (CAS N^{o} 947-84-2) (30 g, 151 mmol, 1.0 equiv) was suspended in toluene (200 ml). The suspension was cooled to T<5° C. before adding DMF (0.2 ml). The mixture was stirred for 10 min at T<5° C. before (COCl)₂ (14.3 ml, 166 mmol, 1.1 equiv) was added dropwise. During the addition gas evolved. The mixture was stirred at RT (overnight). The crude mixture containing biphenyl-2-carbonyl chloride (5) (Yield: 35 g (yellow oil), quantitative) was concentrated and used as such for the next step.

4-Amino-2-chlorobenzoic acid (6) (CAS N^{o} 2457-76-3) (21.6 g, 0.126 mol, 1.0 equiv) was suspended in toluene (100 ml). To the mixture pyridine (57 ml, 0.694 mol, 5.5 equiv) was added and stirred until the mixture was a solution. The solution was cooled to T<5° C. (suspension) and TMS-Cl (27.8 ml, 0.353 mol, 2.8 equiv) was added. This was stirred below 5° C. for 30 min. Subsequently a solution of the aforementioned concentrate (5) (27.9 g from 25 g of 2-phenylbenzoic acid) in toluene (50 ml) was added dropwise. The suspension turned to pink/red. The mixture was stirred for 2.5 hours at a temperature below 5° C. A mixture of conc. HCl (50 ml), demi water (90 ml) and ethanol (90 ml) was added and the temperature rose to 35° C. (thick suspension). The suspension was stirred for 20 min and then heated to 85° C. for 30 min. The mixture was allowed to cool to RT. The solid was filtrated and washed with water/ethanol (1:1 100 ml), water (100 ml) and warm TBME (2×100 ml). The solid was dried in an oven (50° C.) to yield 4-biphenyl-2-ylcarboxamido-2-chlorobenzoic acid (7) (Yield: 31 g (white solid), 69%).

### Analytical Data

¹H-NMR (300 MHz, CDCl₃): 6.99 (d, 2H), 7.40-7.62 (m, 8H), 7.96 (t, 2H)

### B.1.3. Preparation of (S)-tert-butyl 1-(4-biphenyl-2-ylcarboxamido-2-chlorobenzoyl)-3-(hydroxymethyl)-2,3-dihydro-1H-benzo[e][1,4]diazepine-4(5H)-carboxylate (13)

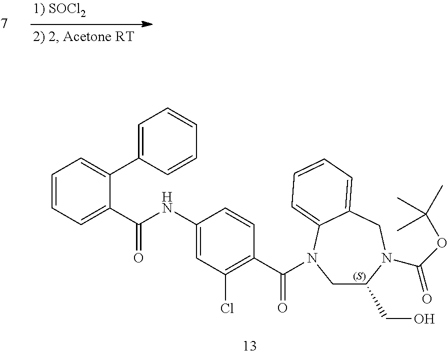

Compound 7 (31 g, 89.8 mmol, 1.0 equiv) was suspended in n-butyl acetate (200 ml). To the suspension thionyl chloride (SOCl₂) (16.3 ml, 224.5 mmol, 2.5 equiv) was added and the mixture was heated to 64° C. for 1.5 hours. The mixture was concentrated and dissolved in dry acetone (50 ml). This was added dropwise to a cooled solution of compound 2 (45 g, 161.7 mmol, 1.8 equiv) in dry acetone (1 L). The ice/water bath was removed and the mixture was stirred at RT (overnight). The mixture was concentrated and purified by column chromatography (silica 4.5 L, heptanes/EtOAc=2/3->2/5) yielding (S)-tert-butyl 1-(4-biphenyl-2-ylcarboxamido-2-chlorobenzoyl)-3-(hydroxymethyl)-2,3-dihydro-1H-benzo[e][1,4]diazepine-4(5H)-carboxylate (13) (Yield: 48 g (white solid), 89%).

### Analytical Data

¹H-NMR (300 MHz, CDCl₃): 1.48 (s, 9H), 3.08 (d, 1H), 3.80 (d, 2H), 4.10 (t, 1H), 4.60 (t, 1H), 4.68 (d, 1H), 5.07 (d, 1H), 6.80 (s, 3H), 7.00 (t, 2H), 7.13 (t, 1H), 7.26 (d, 1H), 7.41-7.62 (m, 8H), 7.86 (d, 1H)

### B.1.4. Preparation of (S)—N-(3-chloro-4-(3-(hydroxymethyl)-2,3,4,5-tetrahydro-1H-benzo[e][1,4]diazepine-1-carbonyl)phenyl)biphenyl-2-carboxamide (14)

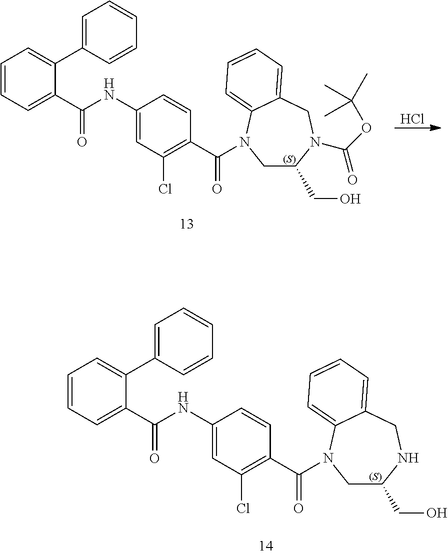

Compound 13 (48 g, 78.4 mmol, 1.0 equiv) was largely dissolved in dioxane (1 L). To the mixture 4N HCl in dioxane (196 ml, 784 mmol, 10.0 equiv) was added and stirred at RT (over week-end). Water (800 ml) was added to the mixture and the pH was adjusted to 7-8 with sat NaHCO₃. The aqueous mixture was extracted with EtOAc (3×750 ml). The combined organic layers were dried (Na₂SO₄) and concentrated, yielding crude (S)—N-(3-chloro-4-(3-(hydroxymethyl)-2,3,4,5-tetrahydro-1H-benzo[e][1,4]diazepine-1-carbonyl)phenyl)biphenyl-2-carboxamide (14) (Yield: 40.7 g (white foam), quantitative). A sample of M001 (2.5 g) was purified by automated column chromatography (EtOAc 100% to EtOAc/MeOH=4/1). The fractions containing 14 were combined and evaporated, dissolved in MeOH an poured into water. The milky solution was partly concentrated and the subsequently was freeze dried overnight, yielding white solid M001 (Yield: 1.5 g, 60%).

### Analytical Data

¹H-NMR (300 MHz, CDCl₃): 2.28 (s, NH,OH) 2.69 (m, 1H), 3.34 (d, 1H), 3.52 (t, 1H), 3.83 (d, 2H), 4.05 (d, 1H), 4.95 (d, 1H), 6.70 (t, 1H), 6.86 (s, 3H), 7.01 (t, 1H), 7.12 (t, 1H), 7.23 (t, 1H), 7.40-7.62 (m, 8H), 7.85 (d, 1H)

### B.1.5. Preparation of (S)-tert-butyl 2-(1-(4-biphenyl-2-ylcarboxamido-2-chlorobenzoyl)-3-(hydroxymethyl)-2,3-dihydro-1H-benzo[e][1,4]diazepin-4(5H)-yl)acetate (15)

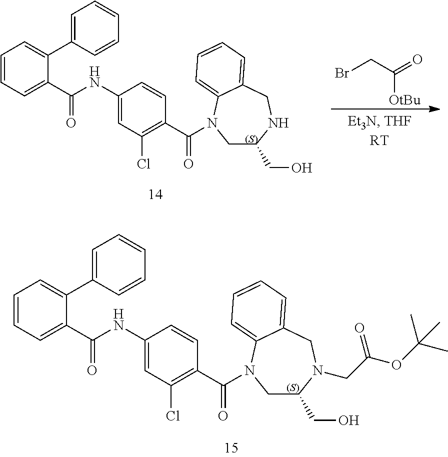

Compound 14 (15 g, 29.3 mmol, 1.0 equiv), Et₃N (8.1 ml, 43.9 mmol, 2,0 equiv) and tert-butyl bromo acetate (5.0 ml, 30.7 mmol, 1.05 equiv) were added to acetonitrile (75 ml). The mixture was stirred at RT (overnight). Water (150 ml) was added to the mixture and this was extracted with EtOAc (150 ml). The organic layer was washed with water (150 ml) and dried (Na₂SO₄). The mixture was concentrated and purified by column chromatography (silica 1 L, heptanes/EtOAc=2/3), yielding (S)-tert-butyl 2-(1-(4-biphenyl-2-ylcarboxamido-2-chlorobenzoyl)-3-(hydroxymethyl)-2,3-dihydro-1H-benzo[e][1,4]diazepin-4(5H)-yl)acetate (15) (Yield: 7 g (white foam), 38%).

### Analytical Data

¹H-NMR (300 MHz, CDCl₃): 2.97 (t, 1H), 3.10 (s, 1H), 3.19 (t, 1H), 3.18 (t, 1H), 3.66 (m, 1H), 3.79 (s, 2H), 3.87 (d, 1H), 4.43 (m, 1H), 4.76 (d, 1H), 6.70 (dd, 2H), 6.89 (q, 1H), 6.98-7.18 (m, 4H), 7.21-7.37 (m, 7H), 7.42 (t, 1H), 7.64 (d, 1H)

### B.2 Synthesis of Final Compounds

### B.2.1. Preparation of (S)—N-(3-chloro-4-(3-oxo-3,4,6,11,12,12a-hexahydro-1H-benzo[e][1,4]oxazino[4,3-a][1,4]diazepine-11-carbonyl)phenyl)biphenyl-2-carboxamide (16)

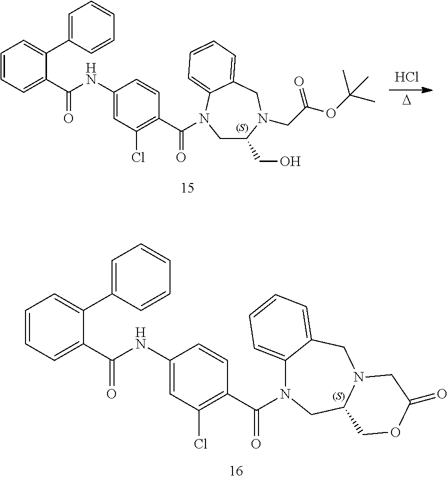

Compound 15 (7 g, 11.18 mmol, 1.0 equiv) was dissolved in dioxane (150 ml). To the mixture 4N HCl in dioxane (28 ml, 111.8 mmol, 10.0 equiv) was added and the mixture was heated to reflux for 1 hour. Added water (100 ml and EtOAc (150 ml) to the mixture, there was some white solid between the layers. After adding sat NaHCO₃ (30 ml) there was still some solid between the layers. The layers were separated and the solid was collected and dried under vacuum (50° C.) to yield compound 16 (470 mg, 99% pure).

The organic layer was dried (Na₂SO₄) and concentrated. The residue was stirred in DCM and filtrated. The filtrate was concentrated to yield compound 16 (2.6 g, yellow foam, 96% pure). This was crystallized in DCM to yield compound 16 (1.7 g, 100% pure). (Total yield: 2.37 g (white solid), 38%).

### Analytical Data

¹H-NMR (300 MHz, DMSO): 1.67 (s, OH), 2.64 (t, 1H), 2.79 (t, 1H), 2.99 (s, 1H), 3.18 (d, 1H), 3.59 (d, 2H), 3.92 (t, 1H), 4.48 (d, 2H1H), 10.44 (s, NH)

### B.2.2. Preparation of (S)-2-(1-(4-biphenyl-2-ylcarboxamido-2-chlorobenzoyl)-3-(hydroxymethyl)-2,3-dihydro-1H-benzo[e][1,4]diazepin-4(5H)-yl)acetic acid (17)

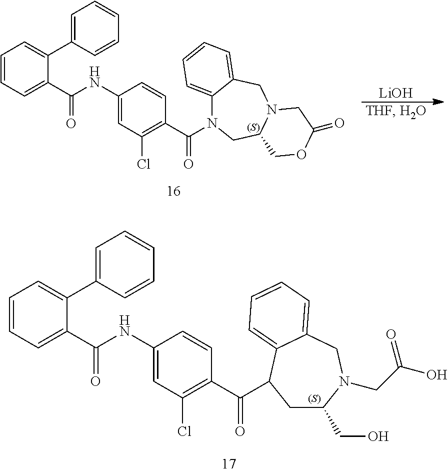

Compound 16 (1.86 g, 3.16 mmol, 1.0 equiv.) was suspended in THF/water (2:1=30 ml). To the suspension LiOH monohydrate (0.27 g, 6.32 mmol, 2.0 equiv) was added (turned into a clear solution) and was stirred o/n at RT. The mixture was concentrated and the gel like substance was dissolved in a mixture of water/MeOH/DCM. The mixture was neutralized to pH 7-8 by 2N HCl. The layers were separated and the aqueous layer was concentrated. The solid was dried in the oven (50° C.). The solid was dissolved in THF/water and concentrated yielding (S)-2-(1-(4-biphenyl-2-ylcarboxamido-2-chlorobenzoyl)-3-(hydroxymethyl)-2,3-dihydro-1H-benzo[e][1,4]diazepin-4(5H)-yl)acetic acid (17) (Yield: 1 g (off-white (glass like) solid), 57%).

### Analytical Data

¹H-NMR (300 MHz, DMSO): 1.67 (s, OH), 2.64 (t, 1H), 2.79 (t, 1H), 2.99 (s, 1H), 3.18 (d, 1H), 3.59 (d, 2H), 3.92 (t, 1H), 4.48 (d, 2H1H), 10.44 (s, NH)

### B.2.3. Preparation of (S)—N-(3-chloro-4-(3-hydroxy-3,4,6,11,12,12a-hexahydro-1H-benzo[e][1,4]oxazino[4,3-a][1,4]diazepine-11-carbonyl)phenyl)biphenyl-2-carboxamide (19)

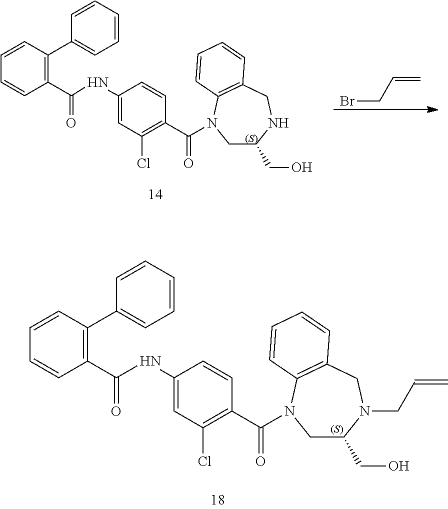

Compound 14 (9.6 g, 18.75 mmol, 1.0 equiv) was dissolved in acetonitrile (200 ml). To the solution K₂CO₃ (10.4 g, 75 mmol, 4.0 equiv) and allyl bromide (1.7 ml, 19.69 mmol, 1.05 equiv) were added and the mixture was heated to reflux for 1 hour. The mixture was allowed to cool to RT (overnight). The mixture was concentrated and the residue suspended in water (100 ml). The aqueous layer was extracted with DCM (2×100 ml). The organic layer was washed with water (100 ml) and dried (Na₂SO₄). The filtrate comprising (S)—N-(4-(4-allyl-3-(hydroxymethyl)-2,3,4,5-tetrahydro-1H-benzo[e][1,4]diazepine-1-carbonyl)-3-chlorophenyl)biphenyl-2-carboxamide (18) was concentrated and used as such in the next step.

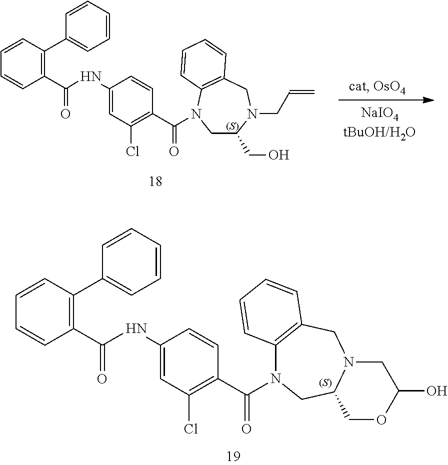

Crude compound 18 (9.4 g, 17.0 mmol, 1.0 equiv) was dissolved in dioxane/THF (2:1, 150 ml). To the solution OsO₄ 4% in water (5.15 ml, 0.85 mmol, 0.05 equiv) was added and stirred for 30 min. Then a solution of NaIO₄ (10.9 g, 51.1 mmol, 3.0 equiv) in water (150 ml) and a solution of NaOAc (3.5 g, 25.5 mmol, 1.5 equiv) in water (50 ml) were added and a suspension was formed. The mixture was stirred at RT (overnight). The suspension was filtered and the amount of THF was evaporated. The remaining mixture was extracted with DCM (300 ml). The organic layer was washed with water (300 ml) and dried (Na₂SO₄). The mixture was concentrated and purified by column chromatography (silica 600 ml, DCM/EtOAc=1/1->100% EtOAc). The crude mixture was crystallized in EtOAc/heptane (heptane added after dissolving in EtOAc). The solid was dissolved in MeOH and poured into water and the formed precipitate was filtered. The solid was dried in the oven (50° C.), yielding (S)—N-(3-chloro-4-(3-hydroxy-3,4,6,11,12,12a-hexahydro-1H-benzo[e][1,4]oxazino[4,3-a][1,4]diazepine-11-carbonyl)phenyl)biphenyl-2-carboxamide (19) (Yield: 2 g (white solid), 21%).

### Analytical Data

¹H-NMR (300 MHz, CDCl₃): 2.40 (dd, 1H), 2.73 (s, 1H), 2.85 (d, 1H), 3.08 (d, 1H), 3.42-3.70 (m, 2H), 3.79 (s, 1H), 4.08-4.25 (m, 1H), 4.77 (dd, 1H), 5.00 (2s, 1H), 6.73 (t, 1H), 6.83 (s, 2H), 6.86 (d, 1H), 7.03 (t, 1H), 7.17 (t, 1H), 7.22 (d, 1H), 7.43 (s, 5H), 7.48-7.61 (m, 3H), 7.84 (d, 1H)

### B.2.4. Preparation of N-[3-chloro-4-({13-oxa-1,9-diazatricyclo[9.4.0.0^{{3,8}}]pentadeca-1,3,5,7-tetraen-9-yl}carbonyl)phenyl]-2-phenylbenzamide (21)

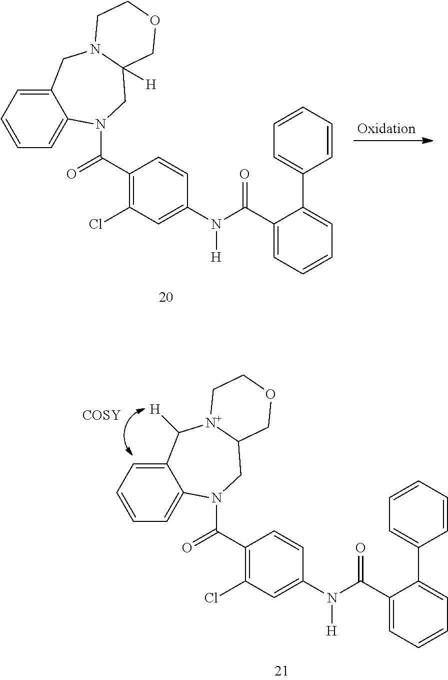

About 100 mg of compound 20 (CAS N^{o} 285571-39-9) was converted into final compound 21 using a biomimetic oxidation reaction using the protocol as described in “Igor Mezine et al., Assessment of Drug Interaction Potential, Genetic Engineering & Biotechnology News, Oct. 15, 2009”. Briefly, compound 21 was synthesized via oxidation of the starting compound 20 using a synthetic porphyrin-transient metal complex, i.e. Fe(III)meso-tetra(pentafluorophenyl)porphine chloride, in the presence of an activator, i.e. cumene peroxide, using peroxide as an oxygen donor. The final compound was isolated and purified using a combination of solid-phase extraction and preparative HPLC, yielding about 2.2 mg of compound 21. (purity >99% by HPLC-UV.

### Analytical Data

¹H-NMR (400 MHz; CD₃OD): 2.76 (m, 2.5H), 2.9 (t, 1.5H), 3.0 (m, 1H), 3.2 (d, 0.8H), 3.4-3.6 (m, 1H), 3.62 (m3.8-3.9 (m. 2.6H), 4.7 (d, 1H), 5.05 (s, 0.3H), 5.17 (s, 1H), 7.9 (d, 1H), 7.95 (d, 1H), 7.2-7.6 (m, 20H), 7.74 (d, 1H).

As shown in the scheme above, a H—H COSY (COrrelated SpectroscopY) spin-spin coupling was observed: coupling 5.17 (s, 1H) with signals at ˜7.5 ppm.

1-16. (canceled)

17. A compound of formula (I) 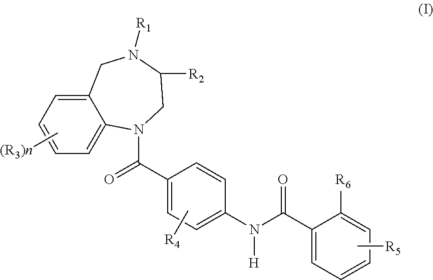
wherein:
n is 0, 1, 2 or 3;
R₁ is hydrogen; C₁₋₆alkenyl optionally substituted by one or more substituents selected from hydroxyl, halogen, nitro, amino, cyano, C₁₋₆alkoxy, hydroxycarbonyl, C₁₋₆alkoxycarbonyl or haloC₁₋₆alkoxy; or C₁₋₆alkyl substituted by one or more substituents selected from hydroxyl, halogen, nitro, amino, cyano, C₁₋₆alkoxy, hydroxycarbonyl, C₁₋₆alkoxycarbonyl or haloC₁₋₆alkoxy;
R₂ is C₁₋₆alkyl substituted by one or more substituents selected from hydroxyl, halogen, nitro, amino, cyano, C₁₋₆alkoxy, hydroxycarbonyl, C₁₋₆alkoxycarbonyl or haloC₁₋₆alkoxy; or
R₁ and R₂ taken together with the atom to which they are attached from a 6 membered heterocycle selected from morpholinyl or thiomorpholinyl substituted with a substituent selected from oxo or hydroxyl at position 6 of said morpholinyl or thiomorpholinyl;
R₃ is independently selected from hydrogen, hydroxyl, halogen, C₁₋₆alkoxy, C₁₋₆alkyl, or haloC₁₋₆alkoxy;
R₄ and R₅ are each independently selected from hydrogen, hydroxyl, halogen, C alkoxy, C₁₋₆alkyl, or haloC₁₋₆alkoxy;
R₆ is independently selected from phenyl or C₆alkyl;
and pharmaceutically acceptable enantiomers, racemates, diastereoisomers, solvates, hydrates, polymorphs and salts thereof.

18. The compound of claim 17, wherein R₁ and R₂ taken together with the atom to which they are attached form a 6 membered heterocycle and wherein the 6 membered heterocycle is selected from the group consisting of piperidinyl, morpholinyl and thiomorpholinyl.

19. The compound of claim 17, wherein n is 1 and R₃ is selected from the group consisting of hydrogen, halogen, and C₁₋₆alkyl.

20. The compound of claim 17, wherein n is 1 and R₃ is hydrogen, halogen or methyl; more particular wherein n is 1 and R₃ is hydrogen.

21. The compound of claim 17, wherein R₄ is selected from the group consisting of hydrogen, hydroxyl, halogen, C₁₋₆alkoxy, and C₁₋₆alkyl.

22. The compound of claim 17, wherein R₄ is selected from the group consisting of hydroxyl, halogen, methyl and methoxy; more particular wherein R₄ is halogen.

23. The compound of claim 17, wherein R₅ is selected from the group consisting of hydrogen, hydroxyl, halogen, C₁₋₆alkoxy, and C₁₋₆alkyl.

24. The compound of claim 17, wherein R₅ is selected from the group consisting of hydrogen, hydroxyl, halogen, and methoxy; more particular wherein R₅ is hydrogen.

25. The compound of claim 17, wherein R₆ is phenyl.

26. A compound of formula (Ic) 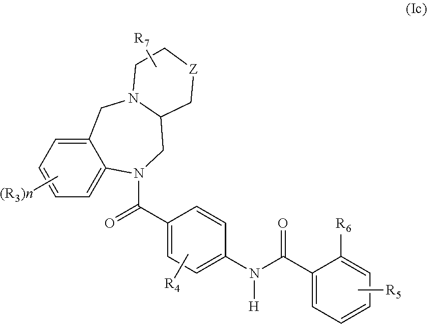
wherein:
n is 0, 1, 2 or 3;
Z is O or S; in particular Z is O;
R₃ is independently selected from hydrogen, hydroxyl, halogen, C₁₋₆alkoxy, C₁₋₆alkyl, or haloC₁₋₆alkoxy;
R₄ and R₅ are each independently selected from hydrogen, hydroxyl, halogen, C₁₋₆alkoxy, C₁₋₆alkyl, or haloC₁₋₆alkoxy;
R₆ is independently selected from phenyl or C alkyl;
R₇ is oxo or hydroxyl at position 6 of said morpholinyl or thiomorpholinyl;
and pharmaceutically acceptable enantiomers, racemates, diastereoisomers, solvates, hydrates, polymorphs and salts thereof.

27. The compound of claim 26, wherein n is 1 and R₃ is selected from the group consisting of hydrogen, halogen, and C₁₋₆alkyl.

28. The compound of claim 26, wherein n is 1 and R₃ is selected from the group consisting of hydrogen, halogen and methyl; more particular wherein n is 1 and R₃ is hydrogen.

29. The compound of claim 26, wherein R₄ is selected from the group consisting of hydrogen, hydroxyl, halogen, C₁₋₆alkoxy, and C₁₋₆alkyl; more particular wherein R₄ is selected from the group consisting of hydroxyl, halogen, methyl and methoxy; more particular wherein R₄ is halogen.

30. The compound of claim 26, wherein R₅ is selected from the group consisting of hydrogen, hydroxyl, halogen, C₁₋₆alkoxy, and C₁₋₆alkyl; more particular wherein R₅ is selected from the group consisting of hydrogen, hydroxyl, halogen, and methoxy; more particular wherein R₅ is hydrogen.

31. The compound of claim 26, wherein R₆ is phenyl.

32. A composition comprising the compound of claim 17 and at least one pharmaceutically acceptable carrier.

33. A composition comprising the compound of claim 26 and at least one pharmaceutically acceptable carrier.

34. A method of treating a vasopressin V2 receptor mediated disorder comprising administering a therapeutically effective amount of the composition of claim 32 to a subject in need.

35. The method of claim 34, wherein the vasopressin V2 receptor mediated disorder is selected from the group consisting of hypertension, hyponatremia, congestive heart failure, cardiac insufficiency, coronary vasospasm, cardiac ischemia, liver cirrhosis, renal vasospasm, renal failure, diabetic nephropathy, polycystic kidney disease, cerebral edema and ischemia, stroke, thrombosis, and water retention.

36. A method of treating a vasopressin V2 receptor mediated disorder comprising administering a therapeutically effective amount of the composition of claim 33 to a subject in need.

37. The method of claim 36, wherein the vasopressin V2 receptor mediated disorder is selected from the group consisting of hypertension, hyponatremia, congestive heart failure, cardiac insufficiency, coronary vasospasm, cardiac ischemia, liver cirrhosis, renal vasospasm, renal failure, diabetic nephropathy, polycystic kidney disease, cerebral edema and ischemia, stroke, thrombosis, and water retention.
